(19) United States
(12) Patent Application Publication
(10) Pub. No.: US 2022/0183784 A1
(43) Pub. Date: Jun. 16, 2022

(54) SURGICAL BASE ASSEMBLIES FOR TRAJECTORY GUIDE SYSTEMS AND ASSOCIATED TRAJECTORY GUIDE SYSTEMS AND METHODS

(71) Applicant: ClearPoint Neuro, Inc., Irvine, CA (US)

(72) Inventors: David John Sayler, Portland, OR (US); Rajesh Pandey, Irvine, CA (US); Maxwell Jerad Daly, Redlands, CA (US); Peter G. Piferi, Orange, CA (US)

(21) Appl. No.: 17/490,745

(22) Filed: Sep. 30, 2021

Related U.S. Application Data
(60) Provisional application No. 63/125,204, filed on Dec. 14, 2020.

(51) Int. Cl. A61B 90/11

(52) U.S. Cl. CPC A61B 90/11

(57) ABSTRACT

A surgical base assembly for an MRI-guided interventional system includes a base body, a plurality of thumbwheels held by the base body and coupled to a respective plurality of threaded members that extend therethrough whereby a user can rotate the thumbwheels to lift the base body to a desired stand-off location relative to patient. The base assembly can couple to a trajectory defining an intrabody trajectory axis and being configured to guide placement of an interventional device in vivo. The threaded members can have sharp tips configured to penetrate bone and/or tissue of the body.

### RELATED APPLICATIONS

This application claims the benefit of and priority to U.S. Provisional Application Ser. No. 63/125,204 filed Dec. 14, 2020, the contents of which are hereby incorporated by reference as if recited in full herein.

### FIELD OF THE INVENTION

The present invention relates generally to medical devices and methods and, more particularly, to medical devices used in image-guided surgeries and methods.

### BACKGROUND

It is often necessary or desirable to mount a trajectory guide frame for MRI-guided surgeries over or even directly on a patient. For example, a frameless stereotactic trajectory guide apparatus may be secured to a patient's skull using bone penetrating screws or the like. Examples of such trajectory guide apparatus are disclosed in U.S. Published Patent Application No. 2009/0112084 A1 and U.S. Pat. No. 9,192,446, the contents of which are hereby incorporated by reference as if recited in full herein.

### SUMMARY

According to embodiments of the present invention, a base assembly is configured to hold a trajectory guide frame and can be held over or be secured to a body of the patient. At least one thumbwheel is held by the base of the base assembly. Each of the at least one thumbwheel receives and threadably engages a threaded member.

The threaded member can each have a first threaded section of a first diameter and a second threaded section of a second diameter.

In some embodiments, the step of securing the base to the skull using a plurality of screws includes driving the screws through the scalp and into the skull.

Embodiments of the invention are directed to base assemblies which may be particularly suitable for engaging a surgical trajectory guide frame. The base assembly has a base body configured to hold a trajectory guide frame. The base body defines an open center patient access aperture. The base assembly has at least one user-input actuator held by the base body and at least one threaded member. One of the at least one threaded member extends through a respective one of the at least one user-input actuator.

The at least one threaded member can have a first threaded section of a first diameter and a second threaded section of a second diameter that is greater than the first diameter.

The second threaded section can threadably engage a respective user-input actuator.

The base body can include spaced apart upper and lower primary surfaces, a first aperture in the upper primary surface and a second aperture in the lower primary surface. The at least one user-input actuator can be held between the upper and lower primary surfaces with an outer perimeter segment thereof extending outward a distance from the base body. The at least one threaded member can reside through the first aperture, then through a center open channel of the at least one user-input actuator, then through the second aperture.

The at least one user-input actuator can be provided as a plurality of spaced apart user-input actuators. The at least one threaded member is a plurality of threaded members, one extending through each of the user-input actuators.

The at least one user-input actuator is a thumbwheel.

The at least one threaded member can have a head, an intermediate segment with external threads, and a shaft segment under the intermediate segment that merges into a lower end portion comprising external threads and a sharp tip configured to self-tap into bone.

The base assembly can also include a nut coupled to each of the at least one user-input actuator. The nut can have internal threads that engage external threads of a respective threaded member whereby the base body can be movable to a stand-off position while the threaded member remains in a fixed position and coupled to the base body.

The base body can have an open center patient access port. The at least one user-input actuator is a plurality of user-input actuator residing at spaced apart locations of the base body.

The at least one user-input actuator can be a plurality of user-input actuators. Each of the plurality of user-input actuators can have a center through channel with a first segment residing under an upper primary surface of the base body that has a larger diameter than a second segment residing beneath the first segment. The base assembly can have a plurality of nuts with internal threads, one nut held inside the first segment of each of the plurality of user-input actuators.

The shaft segment is devoid of threads and extends a distance beneath the intermediate segment.

The base body can be a monolithic molded body that is non-ferromagnetic.

The at least one threaded member can be a monolithic body of non-ferromagnetic metal.

The base body can have a pair of yoke arms and an open center patient access port. The pair of yoke arms can be spaced apart on opposing sides of the patient access port.

Other embodiments are directed to a trajectory guide system. The system includes: a base assembly with a base body coupled to a plurality of threaded members and a plurality of thumbwheels, one threaded member of the plurality of threaded members extending through one thumbwheel of the plurality of thumbwheels. The thumbwheels threadably engage the threaded members and a trajectory guide coupled to the base assembly. The trajectory guide is operable to move relative to the base assembly to position a trajectory axis to a desired intrabody trajectory to guide placement of a surgical device in vivo.

Each of the plurality of threaded members can have first and second longitudinally spaced apart threaded segments of different diameters.

Each of the threaded members can have a head that merges into an intermediate segment with external threads that merges into a shaft segment that merges into a lower end portion comprising external threads and a sharp tip.

The system can have a plurality of nuts, one coupled to each of the threaded members. The nut can have internal threads that engage external threads of a respective threaded member whereby the base body is movable to a stand-off position while the threaded member remains in a fixed position and coupled to the base body.

The plurality of threaded members can be four. Each threaded member can have a first segment of a greater diameter than a second segment and each of the first and second segments comprises external threads. Each threaded member has a sharp tip configured to pierce through a scalp.

The thumbwheels can cooperate with the threaded members to provide a stand-off distance of the base body that is selectively adjustable relative to a distal tip of the threaded members.

Yet other embodiments are directed to methods of mounting an image-guided component on or adjacent a subject. The methods include: providing a surgical base assembly with a plurality of spaced apart threaded members and a plurality of user-input actuators; driving a head of each of the threaded members to extend the threaded members to a first position outside a bottom primary surface of the base assembly; and rotating the user-input actuators about the threaded members to lift or lower the base assembly relative to the subject to a stand-off position while the extended threaded members remain in the first position.

The surgical base assembly can have a center patient access port and pockets in the base body of the base assembly holding the user input actuators, and the user input actuators are thumbwheels.

The method can include attaching a trajectory guide to the surgical base assembly before, during or after the rotating.

The threaded members can have external threads having a first diameter proximate a sharp tip on distal end portion opposing the head. The threaded members can have an intermediate segment with external threads of a second diameter that is greater than the first diameter. The rotating the user-input actuators can be carried out by rotating the user-input actuators about the external threads of the intermediate segment to lift the base assembly.

The driving can be carried out to self-tap a distal end portion of the threaded members into a target subject, such as a skull, to define the first position, wherein the rotating is carried out while the sharp tip is anchored in the subject.

Further features, advantages and details of the present invention will be appreciated by those of ordinary skill in the art from a reading of the figures and the detailed description of the embodiments that follow, such description being merely illustrative of the present invention.

### BRIEF DESCRIPTION OF THE DRAWINGS

FIG. 1 is a top perspective view of a base assembly according to embodiments of the present invention.

FIG. 2 is an exploded, top perspective view of the base assembly of FIG. 1.

FIG. 3 is a side view of an example threaded member of the base assembly shown in FIG. 1 according to embodiments of the present invention.

FIG. 4 is a section view of the base assembly shown in FIG. 1.

FIG. 5 is an enlarged front view of the base assembly shown in FIG. 1 in position on a patient according to embodiments of the present invention.

FIG. 6 is a side perspective view of a surgical assembly comprising the base assembly of FIG. 1 coupled to a trajectory guide frame assembly according to embodiments of the present invention.

FIG. 7A is a side perspective view of the surgical assembly shown in FIG. 6 and mounted on a patient according to embodiments of the present invention.

FIG. 7B is an enlarged view of the device shown in FIG. 7A.

FIG. 8A is a side perspective view of a surgical support system comprising the base assembly of FIG. 1 in position over a patient according to embodiments of the present invention.

FIG. 8B is a side perspective view of the surgical support system shown in FIG. 8A positioned in a different configuration from that shown in FIG. 8A according to embodiments of the present invention.

FIG. 9 is a flow chart of example actions that can be carried out according to embodiments of the present invention.

FIGS. 10A-10C are side perspective views of an example series of Step 1 actions for securing the threaded member to a skull of a patient according to embodiments of the present invention.

FIGS. 11A-11D are side perspective views of an example series of Step 2 actions for lifting/moving the base upward away from the skull according to embodiments of the present invention.

### DESCRIPTION OF EMBODIMENTS OF THE INVENTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

The term “fiducial marker” refers to a marker that can be electronically identified using image recognition and/or electronic interrogation of image data, including, for example, MRI image data and/or CT image data. The fiducial marker can be provided in any suitable manner, such as, but not limited to, a geometric shape of a portion of the tool, a component on or in the tool, a coating or fluid-filled component or feature (or combinations of different types of fiducial markers) that makes the fiducial marker(s) CT and/or MRI-visible with sufficient signal intensity (brightness) or generates a “void” or dark space for identifying location and/or orientation information for the tool and/or components thereof in space.

The term “MRI scanner” refers to a magnetic resonance imaging and/or NMR spectroscopy system. As is well known, MRI scanners include a low field strength magnet (typically between about 0.1 T to about 0.5 T), a medium field strength magnet, or a high-field strength super-conducting magnet, an RF pulse excitation system, and a gradient field system. MRI scanners are well known to those of skill in the art. Examples of commercially available clinical MRI scanners include, for example, those provided by General Electric Medical Systems, Siemens, Philips, Varian, Bruker, Marconi, Hitachi and Toshiba. The MRI systems can be any suitable magnetic field strength, such as, for example, about 1.5 T or about 3.0 T, and may include other high-magnetic field systems between about 2.0 T-10.0 T.

The term “MRI visible” means that the device is visible, directly or indirectly, in an MRI image. The visibility may be indicated by the increased SNR of the MRI signal proximate the device.

The term “MRI compatible” means that the so-called component(s) is suitable for use in an MRI environment and as such is typically made of a non-ferromagnetic MRI compatible material(s) suitable to reside and/or operate in or proximate a conventional medical high magnetic field environment. The “MRI compatible” component or device is “MR safe” when used in the MRI environment and has been demonstrated to neither significantly affect the quality of the diagnostic information nor have its operations affected by the MR system at the intended use position in an MR system. These components or devices may meet the standards defined by ASTM F2503-05. See, American Society for Testing and Materials (ASTM) International, Designation: F2503-05. Standard Practice for Marking Medical Devices and Other Items for Safety in the Magnetic Resonance Environment. ASTM International, West Conshohocken, Pa., 2005.

Embodiments of the present invention are directed to base assemblies of trajectory guide frames (and systems and methods including the same) for image-guided surgeries such as CT or MRI-guided surgeries for medical interventions. Example medical surgeries and interventions are discussed in in U.S. Published Patent Application No. 2009/0112084 A1, U.S. Published Patent Application No. 2019/0346576, U.S. Pat. Nos. 9,192,446, 9,891,296, 10,105,485, and 10,576,247, which are hereby incorporated herein by reference in their entireties.

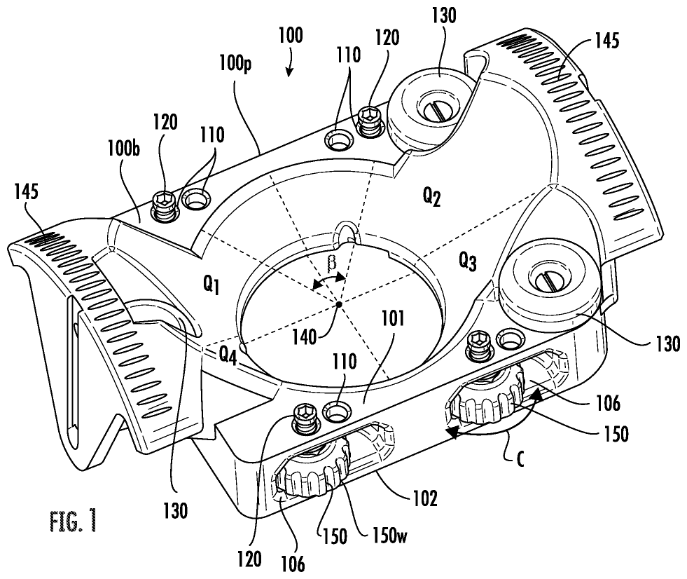

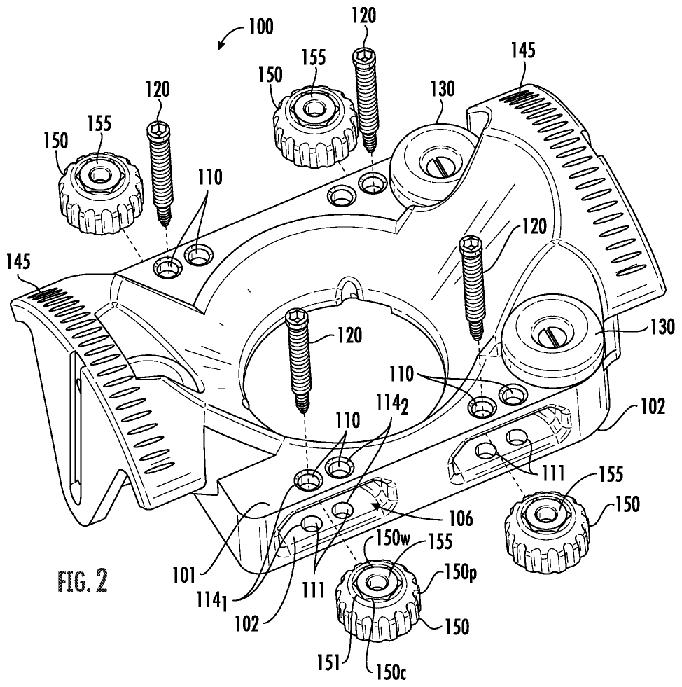

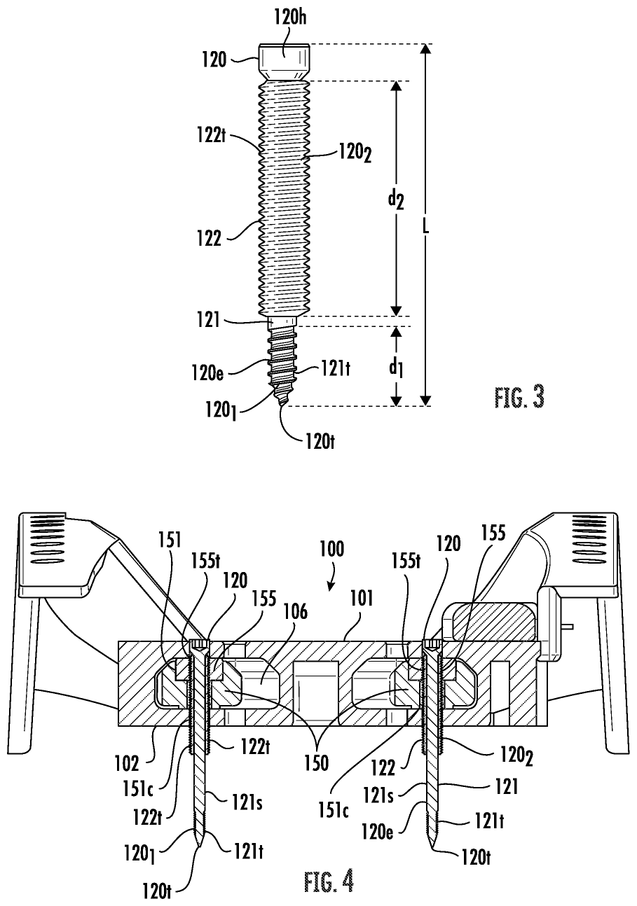

Referring to FIGS. 1 and 2, an example base assembly 100 providing an access port 140 is shown. The base assembly 100 comprises a base body 100b. The base assembly 100 includes at least one user input actuator 150, shown as a thumbwheel 150w. The base assembly 100b also includes a threaded member 120 extending through each of the at least one user-input actuator 150. The threaded member 120 threadably engages the user input actuator 150 and has a lower end portion 120e with a tip 120t (FIG. 3) that extends under and outside the base body 100b.

In the embodiment shown in FIGS. 1 and 2, there are a plurality of (shown as four) user input actuators 150 spaced apart about an outer perimeter portion 100p of the base body 100b.

The base body 100b can be a multiple component device or a unitary monolithic single piece device. The base body 100b comprises an upper primary surface 101 and a bottom primary surface 102.

The base assembly 100 can be used for any suitable procedure and is not limited to surgical image-guided navigation systems. The base assembly 100 can be used with a patient, or object or any target subject. Where used for patients, the patient can be human or an animal for research or veterinarian uses.

A through aperture 110 can be formed through the upper primary surface 101 aligned with a through aperture 111 formed through the bottom primary surface 102. The user input actuator 150 resides in a pocket 106 between the through apertures 110, 111, under the upper primary surface 101 and above the bottom primary surface 102. The threaded member 120 extends through both apertures and the user input actuator 150. The threaded member 120 threadably engages the user input actuator 150.

In the embodiment shown, there are a plurality of spaced apart pairs of aligned apertures 110, 111 that are spaced apart about the patient access port 140. In some embodiments, at least one pair of aligned apertures 110, 111 can reside in the base body 100b aligned with different quadrants Q1, Q2, Q3, Q4 of the patient access port 140, optionally with one pair of apertures 110, 111 in each quadrant residing at an angular extent 13 from each other, measured from a center of the patient access port 140, that is in a range of about 45 degrees to about 120 degrees.

Referring to FIGS. 2 and 4, the base assembly 100 can also include at least one threaded nut 155 affixed to a longitudinally extending inner wall 151 of the user input actuator 150 that surrounds an open (through) center channel 151c. The nut 155 can be positioned at a top portion of a larger diameter portion of the open center channel 151c. The larger diameter open channel 151c can merge into a smaller diameter portion of the open center channel 151c. As shown, the user input actuator 150 is coupled to the nut 155. The nut 155 comprises internal threads 155t that matably engage with external threads 122t of the threaded member 120. However, in other embodiments, the inner wall 151 of the actuator 150 can directly provide the threads and a nut 155 is not required.

The pairs of aligned apertures 110, 111 can include first and second sets 114₁, 114₂ of aligned apertures 110, 111 that are closely adjacently positioned. The upper apertures 110 of the sets 114₁, 114₂ reside above and the lower apertures 111 of the respective sets 114₁, 114₂ reside below a single pocket 106 to provide a back-up (rescue) pair of aligned apertures 110, 111, in case the threaded member 120 breaks or strips in one set such as the first set 114₁, a new threaded member 120 and/or nut 155 can be placed in the other, the second set 114₂ and the base assembly 100 can still be used for the medical procedure.

As shown, the base body 100b can have at least one pocket 106 that is open to an outer perimeter 100p of the base body 100b. The at least one pocket 106 resides between the upper primary surface 101 and the bottom primary surface 102. Each pocket 106 can hold a respective actuator 150. A portion of an outer perimeter 150p of the actuator 150 can extend beyond the pocket 106, a distance outward from the base body 100b. As shown in FIG. 1, an exposed circumferential extent C of the actuator(s) 150 can be in a range of 90-180 degrees.

The base assembly 100 can include a plurality of spaced apart fiducials 130, shown as annular fiducials. The base body 100b can have an open center port 140 defining a patient access aperture. The base body 100b can comprise first and second yoke arms 145 that are spaced apart and face each other across the center port 140.

The base body 100b, threaded member(s) 120, actuator(s) 150 and nut(s) 155, where used, can all be MRI-compatible and formed of non-ferromagnetic materials.

Referring to FIGS. 3 and 4, the threaded member 120 can have a head 120h and a longitudinally spaced apart, opposing end, which can be shaped as a sharp tip 120t. The threaded member 120 can have a first segment 120₁ with a first outer diameter that longitudinally extends a length L between the head 120h and the tip 120t, with a threaded portion at the lower end portion 120e extending a first (sub-length) distance d1. The threaded member 120 also includes a second segment 120₂ with a second outer diameter that longitudinally extends a second (sub-length) distance d2. In the example embodiment shown, the first diameter is less than the second diameter and the first distance d1 is less than the second distance d2. The first distance d1 can be about 50% or less of the second distance d2, such as in a range of 0.1× to 0.5× the second distance. However, the opposite can be true, with the second distance d2 being less than the first distance d1.

The threaded member 120 can be provided as a unitary body of machined non-ferromagnetic, surgical metal. In other embodiments, the threaded member 120 can be provided as an outer member providing the larger diameter threaded segment affixed to an inner member providing the lesser diameter threaded segment. An example material comprises or is titanium.

The threaded member 120 can have a distal end portion 121 with a first externally threaded segment 121t and with another adjacent second shaft segment 121s that is devoid of threads. The shaft segment 121s can optionally be configured with a continuous (smooth) outer surface. The threaded member 120 can have an intermediate portion 122 residing between the head 120h and the distal end portion 121 that defines the second segment 120₂ of the threaded member 120 and provides the threaded segment 122t of greater diameter than the threaded segment 121t of the distal end portion 121. The shaft segment 121s can reside between the threaded segment 122t of greater diameter and the threaded segment 121t of lesser diameter.

The threaded member 120 can, in some embodiments, can have an overall length in a range of 0.5-3 inches, more typically in a range of about 0.75 inches to about 2 inches. The threaded sub-segment 121t can extend a distance d1 that is in a range of about 0.15 inches to about 0.5 inches. The threaded segment 122t can have a length that defines the second distance d2 of the second segment 120₂, which can be in a range of 0.25-2 inches, more typically about 0.5 inches to about 1.5 inches.

Referring to FIGS. 3 and 4, the distal end portion 121 of each threaded member 120 can conically taper down to a relatively sharp tip 120t. According to some embodiments, the tips 120t are capable of piercing and penetrating through a scalp upon application of a pressing load, optionally by manually pressing via a hand.

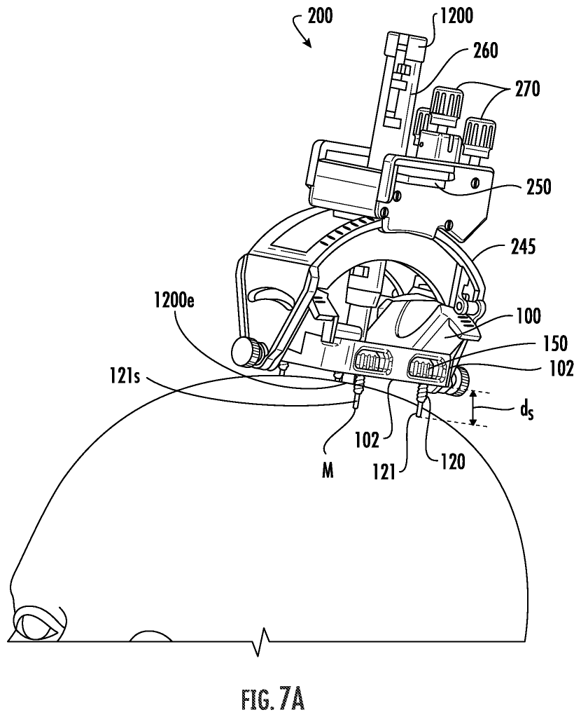

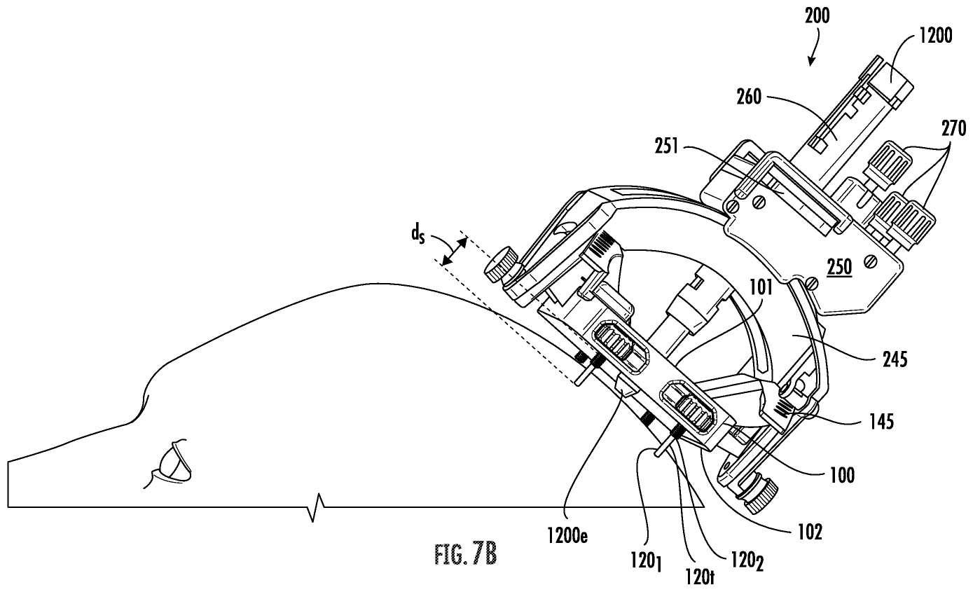

As shown in FIGS. 4 and 5, a lower portion of the second segment 120₂ can extend outside the bottom surface 102 of the base body 100b. A user can rotate the threaded member 120 a first direction (clockwise or counterclockwise) to drive/extend a lower end portion 120e out from the bottom 102 of the base body 100b, optionally to anchor and couple the threaded segment 121t at the distal end portion 121 and/or tip 120t to a scalp and/or skull of a patient (FIGS. 5, 7A, 7B).

In some embodiments, when the threaded member 120 is rotated a first direction, e.g., clockwise, the lower end 120e engages with the skull and threads itself/self-tapping into the skull. This is the bone screw section of the threaded member 120 which can provide the actual physical attachment to the skull. The actuators 150 are engaged with threads 122t of respective user actuators. When the actuators 150 are rotated, they lift the base body 100b off-of/away from the skull while the lower end of the threaded member(s) 120e holds the base body 100b to the skull. The threaded segment 122t cooperates with the corresponding actuator 150 to pull the base body 100b away from the skull. The counteracting forces stabilize the base body 100b.

A user can rotate the actuator 150 which engages the external threads 122t of the second segment 120₂ to lift or lower the base body 100b to a desired stand-off position (ds) as shown in FIG. 5.

The base assembly 100 can be used in two ways, depending on whether the tip 120t of the threaded member 120 is screwed into the patient as an anchor or if the tip 120t is contacting/resting against the patient as a standoff without actually attaching to the skull or other target ROI.

When used in the first manner, the threaded member 120 is screwed into the patient and the user input actuator 150 can then be rotated (about the now fixed screw) to adjust the height of the base assembly over the patient. When used in the other manner, the threaded member 120 isn't embedded in the patient, it's just functioning as a standoff. However, the threaded member 120 can optionally be screwed relative to the user input actuator 150 (nut 155) to pre-position the threaded member 120 in the open channel 150c of the user input actuator 150. The user input actuator 150 can thereafter be used to further adjust a length of the threaded member 120 that extends out from the bottom 102 of the base assembly 100.

The user input actuator 150 is used to adjust the distance between the screw tip 120t and the base assembly 100. That is, the user input actuator 150 is configured to change an extension length of the threaded member 120 relative to a bottom 102 of the base assembly 100. The base assembly 100 is configured to translate the threaded member relative to the base and/or the thumbwheel to a first position to position the tip 120t outside the base assembly 100 at a desired fixed position then use the user input actuator 150 to translate the base assembly 100 relative to the tip 120t to define a standoff distance of the base assembly 100 relative to the tip 120t while the 120t tip remains at the fixed position.

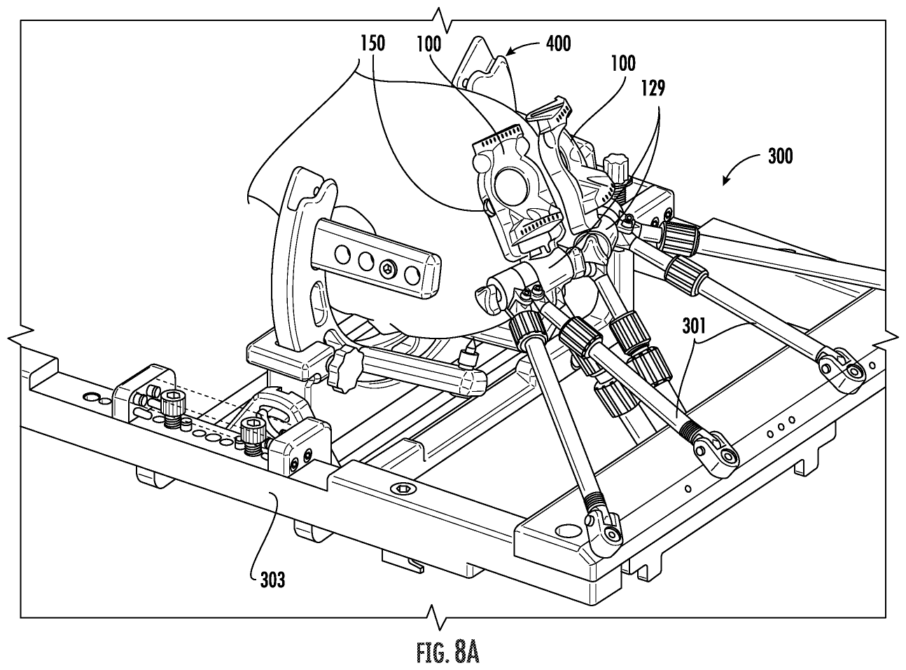

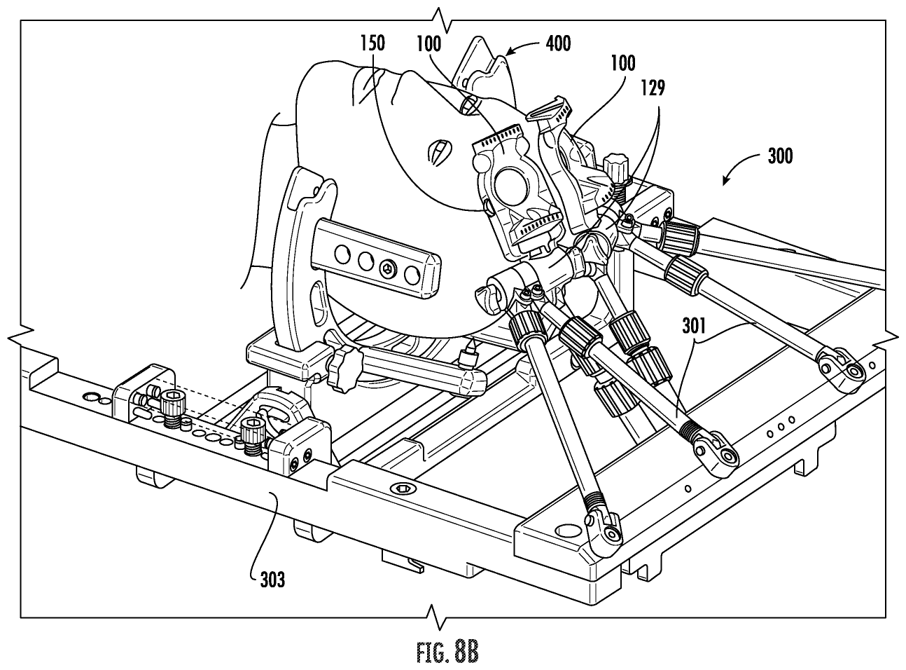

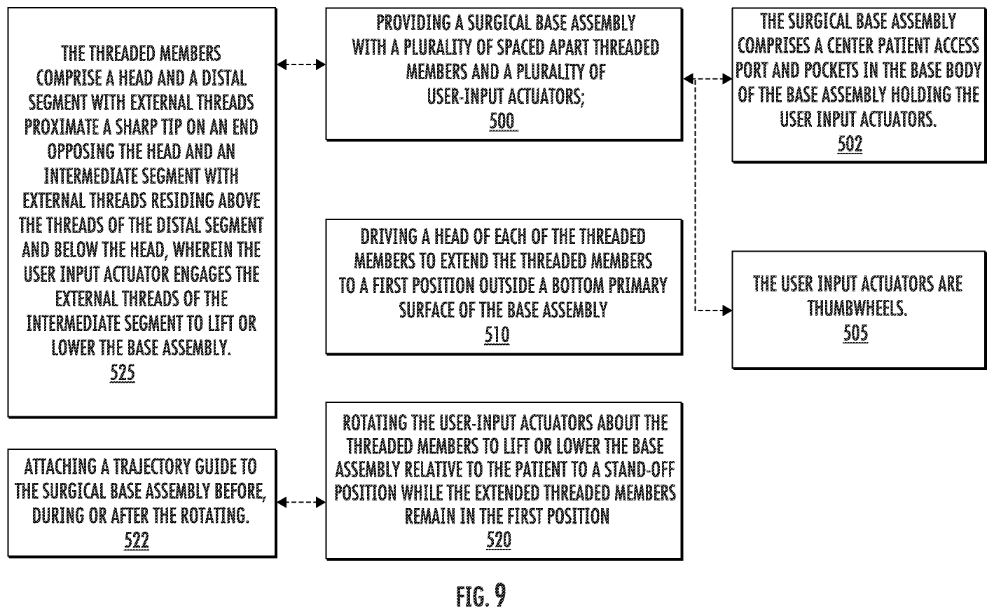

In some embodiments, the tip 120t of the threaded member 120 is not required to anchor to the patient but the actuator 150 can be rotated to lift or lower the bottom 102 of the base assembly 100 to a desired stand-off position (ds) relative to the patient (FIGS. 8A, 8B, for example). The tip 120t can remain in position while the actuator 150 rotates about the threaded segment 120₂ to lift or lower the base body 100b to the desired stand-off position.

Referring to FIG. 5, the user input actuator 150 can cooperate with the second segment 120₂ of the threaded member 120 to lift the base body 100b in an adjustable range to thereby provide adjustability in the stand-off distance ds. The range can be about 0.1 inches to about 1 inch, in some embodiments. The adjustability allows a user to position the patient access port 140 at a desired location relative to the patient which positions the lower end 1200e of the trajectory guide 1200 at a desired position below the bottom surface 102 of the base body 100b, adjacent a patient entry location (FIG. 7A).

Advantageously, in some embodiments, the threaded member(s) 120 can be configured to provide a dual functionality: (i) to be able to move the bottom 102 of the base body 100b away from the head of the patient to a desired stand-off position; and (ii) to concurrently reside against, optionally anchor/attach to a skull and/or scalp of a patient. Thus, the number of threaded members 120 can be reduced from mount configurations requiring different pins for different purposes, such as from 7 to 4 while still providing the same fixation locations and adjustment for a base stand-off position (FIG. 7A) relative to the head of the patient in contrast to the mount configurations disclosed in U.S. Pat. No. 9,192,446. The reduction in numbers of different components previously required to provide the same fixation and standoff functions can reduce time and facilitate installation thereby reducing preop preparation time for different medical procedures.

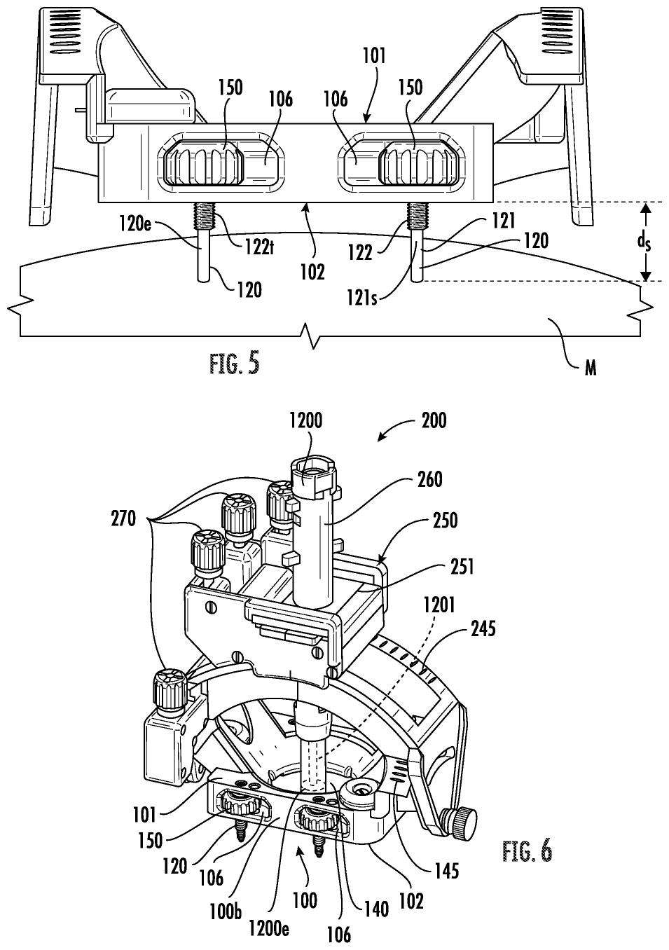

With reference to FIGS. 6, 7A and 7B, a trajectory guide system 200 according to embodiments of the present invention is shown. The system 200 includes a tubular trajectory guide 1200 held by a tower 260, a platform 250, an X-Y table 251, actuators 270, a yoke 245, and the base assembly 100. The trajectory guide 1200 has a guide lumen 1201 configured to receive an interventional device that can enter the patient via a defined trajectory provided by the trajectory guide 1200 of the system 200.

The platform 250 may be movably mounted on the yoke 245 to rotate about on pitch axis. The yoke 245 may in turn be movably mounted on the base portion 1110 to rotate or pivot about a roll axis transverse (in some embodiments, perpendicular) to the pitch axis The platform 250 may be further configured to selectively translate the trajectory guide 1200 along each of an X-axis and a Y-axis provided by an X-Y table 251, relative to the yoke 245. Various local and/or remote control mechanisms and/or actuators can be coupled to the actuators 270 of the trajectory guide system 200 to provide trajectory adjustments.

The base assembly body 100b may be formed of any suitable material, and for MRI uses, can be configured with an MRI-compatible and/or MRI safe material, such as any non-ferromagnetic material and is typically a substantially rigid polymeric material. The base body 100b may be formed of molded polycarbonate, for example.

As discussed below, when used according to some embodiments, when installed, each threaded member 120 can have the tip 120t and a portion of the threaded segment 121t that is embedded in the patient's skull M (FIG. 5) (or other tissue, when the base assembly 100 is used for a procedure targeting another part of the patient) and a portion that protrudes above the patient's skull M (or other targeting entry location). According to some embodiments, the lead end of the screw thread 120t is self-tapping.

Referring to FIGS. 8A, 8B, in some embodiments, the base assembly 100 can be used with a support system 300 comprising a set of length adjustable legs 301. The support system 300 can be configured to be held by a frame 303 that couples to a scanner bed. The base assembly 100 can comprise a sleeve 129 that couples to the support system 300. FIG. 8A illustrates the patient with the head facing down and FIG. 8B illustrates the patient with the head facing up. The support system 300 can cooperate with a head fixation system 400. As shown, two base assemblies 100, each with at least one user input actuator 150 are concurrently used for a bilateral procedure. Further details of the example support system 300 can be found in U.S. Provisional Patent Application Ser. No. 62/968,210, filed Jan. 31, 2020, the contents of which are hereby incorporated by reference as if recited in full herein.

Although described for use with a head (e.g., for brain surgeries), according to other embodiments, the system 200 may be used to operatively secure the trajectory guide 1200 to a selected location on the patient other than the skull M.

In some embodiments, the base assembly 100 is pre-pressed onto the scalp to drive the pin tips 120t down into the scalp all the way to (and in abutment with) the skull M or to a position proximate the skull M.

The yoke 245, tower 260, platform 250, and targeting guide member 1200 can be mounted on the base assembly 100 (before or after mounting the base assembly 100 on the skull M).

In some embodiments, the procedure is continued using a burr hole formed in the skull M as an access portal to the brain and employing the trajectory guide system 200 affixed to the skull of the patient. The trajectory guide system 200 may allow the operator to align an access path trajectory to a predetermined internal target site, such that the interventional/surgical device/lead, therapy, etc. will be delivered to or a sample obtained (e.g., aspirated) from the target site following the desired trajectory (e.g., a planned trajectory line) through the cranial tissue. This trajectory goes through an entry location point. The interventional device (e.g., probe, lead or the like) can be advanced through the guide cannula 1200, into the head and to or proximate the target point.

An incision may be formed in the scalp through the access opening before or after the base assembly 100 has been installed on the skull M. A burr hole may be formed (e.g., by drilling) in the skull M before or after mounting the base assembly 200 on the skull M.

In some embodiments, the trajectory guide system 200 is mounted on the skull M and the trajectory guide 1200 is used as a drill guide for a drill bit that is inserted through the access opening under the access port 140 to form the burr hole. Exemplary methods and apparatus for using a trajectory guide system for forming a burr hole are disclosed in U.S. patent application Ser. No. 13/781,049, filed Feb. 28, 2013, the disclosure of which is incorporated herein.

According to some embodiments, by spacing the base assembly 100 (i.e., the bottom surface 102 of the base assembly body 100b) off of the scalp, the base assembly 100 is provided with a stable attachment and is prevented from placing pressure on the scalp. Such pressure is undesirable as it may make the base assembly 100 and/or the trajectory guide system 200 unstable, and may compress the scalp or other tissue, causing necrosis. Moreover, it is not necessary to peel back a large area of the scalp to expose the skull M to directly mate the base assembly to the skull M.

The base assembly 100 can also serve to stabilize the trajectory guide system 200 during and after mounting. During installation, the base assembly 100 can set the orientation with respect to the skull M so that the insertion depth of each threaded member 120 into the skull M is correspondingly set. Thus, it is not necessary to carefully control the driven depth of the threaded members 120 to avoid misaligning or cocking the base assembly body 100b with respect to the skull M. Each of the threaded members 120 can be driven to the depth appropriate to achieve an appropriate interlock.

Advantageously, the base assembly 100 may be suitably mounted on a skull area of substantially any curvature. The base assembly 100 can be mounted on a skull with the threaded members 120 extending through the scalp rather than requiring the scalp first be cut or removed to allow the base assembly 100 to interface directly with the skull M.

According to some embodiments, the length of the shaft 121s that defines a skull embedded section is in the range of from about 2 mm to 10 mm.

It will be appreciated that aspects of the present invention can be used with or incorporated into trajectory guide frames of other types and configurations.

The trajectory guide systems 200 of the present invention can be provided as a sterile kit (typically as single-use disposable hardware) or in other groups or sub-groups or tools or even individually, typically provided in suitable sterile packaging. The tools can also include a marking grid (e.g., as disclosed in U.S. Published Patent Application No. 2009/0177077 and/or U.S. Published Patent Application No. 2009/0171184). Certain components of the kit may be replaced or omitted depending on the desired procedure. Certain components can be provided in duplicate for bilateral procedures.

Trajectory guide systems and base assemblies in accordance with embodiments of the invention may be used to guide and/or place diagnostic or interventional devices and/or therapies to any desired internal region of the body or object using image guided surgeries, e.g., CT or MRI and/or in an MRI scanner or MRI interventional suite. The object can be any object and may be particularly suitable for animal and/or human subjects.

In some embodiments, the guide apparatus is used to place implantable DBS leads for brain stimulation, typically deep brain stimulation. In some embodiments, the guide apparatus can be configured to deliver tools or therapies that stimulate a desired region of the sympathetic nerve chain. Other uses inside or outside the brain include stem cell placement, gene therapy or drug delivery for treating physiological conditions. Some embodiments can be used to treat tumors. Some embodiments can be used for RF ablation, laser ablation, cryogenic ablation, etc. In some embodiments, the interventional tools can be configured to facilitate high resolution imaging via intrabody imaging coils (receive antennas), and/or the interventional tools can be configured to stimulate local tissue, which can facilitate confirmation of proper location by generating a physiologic feedback (observed physical reaction or via fMRI).

In some embodiments, the trajectory guide system and base assembly are used for delivering bions, stem cells or other target cells to site-specific regions in the body, such as neurological target and the like. In some embodiments, the guide apparatus is used to introduce stem cells and/or other cardio-rebuilding cells or products into cardiac tissue, such as a heart wall via a minimally invasive MRI-guided procedure, while the heart is beating (i.e., not requiring a non-beating heart with the patient on a heart-lung machine). Examples of known stimulation treatments and/or target body regions are described in U.S. Pat. Nos. 6,708,064; 6,438,423; 6,356,786; 6,526,318; 6,405,079; 6,167,311; 6,539,263; 6,609,030 and 6,050,992, the contents of which are hereby incorporated by reference as if recited in full herein.

Generally stated, some embodiments of the invention are directed to MRI interventional procedures including locally placing interventional tools or therapies in vivo to site-specific regions using an MRI system. The interventional tools can be used to define an MRI-guided trajectory or access path to an in vivo treatment site.

In some embodiments, MRI can be used to visualize (and/or locate) a therapeutic region of interest inside the brain or other body locations, and to visualize (and/or locate) an interventional tool or tools that will be used to deliver therapy and/or to place a chronically implanted device that will deliver one or more therapies. Then, using the three-dimensional data produced by the MRI system regarding the location of the therapeutic region of interest and the location of the interventional tool, the system and/or physician can make positional adjustments to the interventional tool so as to align the trajectory of the interventional tool, so that when inserted into the body, the interventional tool will intersect with the therapeutic region of interest. With the interventional tool now aligned with the therapeutic region of interest, an interventional probe can be advanced, such as through an open lumen inside of the interventional tool, so that the interventional probe follows the trajectory of the interventional tool and proceeds to the therapeutic region of interest.

FIG. 9 illustrates example actions of methods for installing a base assembly that can be carried out according to embodiments of the present invention. A surgical base assembly with a plurality of spaced apart threaded members and a plurality of user-input actuators is provided (block 500). A head of each of the threaded members can engage a driver to extend the threaded members to a first position relative to a patient (block 510). The user-input actuators can be rotated about the threaded members to lift (or lower) the base assembly away from (or toward) the patient to a stabilized position, optionally a stand-off position while the extended threaded members remain in the first position (block 520).

The surgical base assembly can further comprise a center patient access port and pockets holding the user input actuators (block 502).

The user input actuators can be thumbwheels (block 505).

The methods can include attaching a trajectory guide to the surgical base assembly before, during or after the rotating (block 522).

The threaded members can comprise a head and external threads proximate a sharp tip on an end opposing the head and an external intermediate segment with external threads residing above the threads proximate the sharp (self-tapping) tip of and below the head. The user input actuator engages the external threads of the intermediate segment to pull/lift the base assembly (block 525).

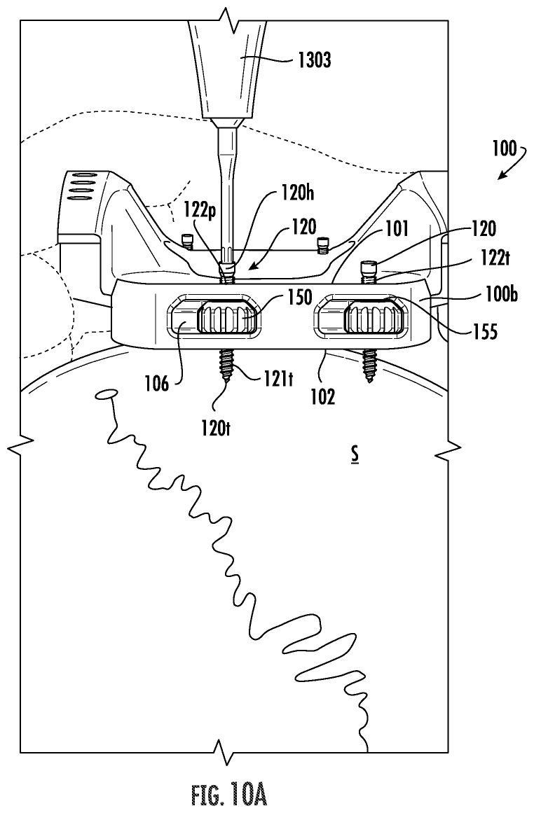

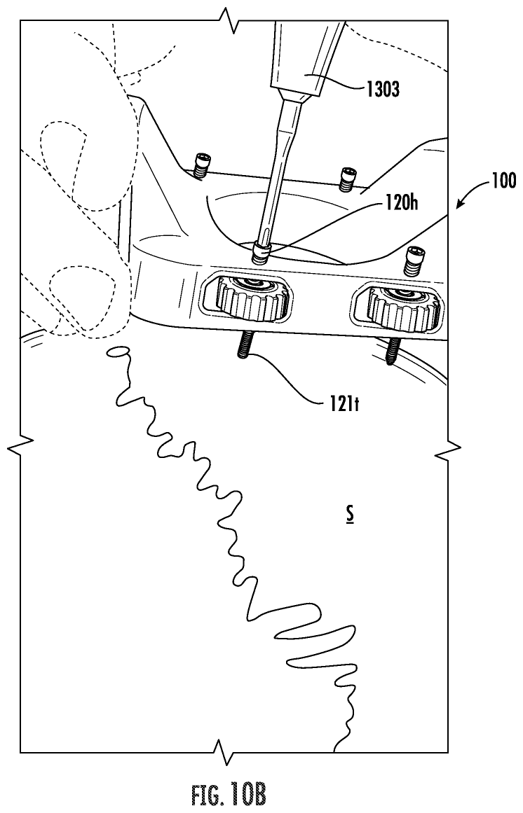

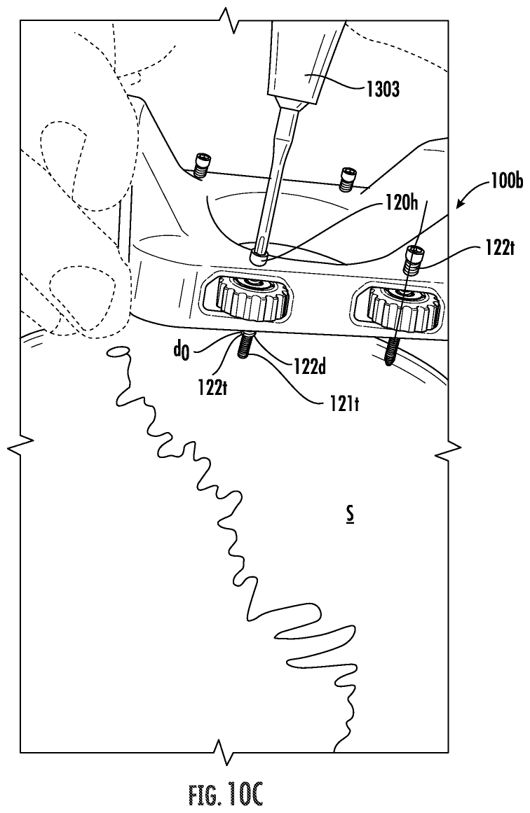

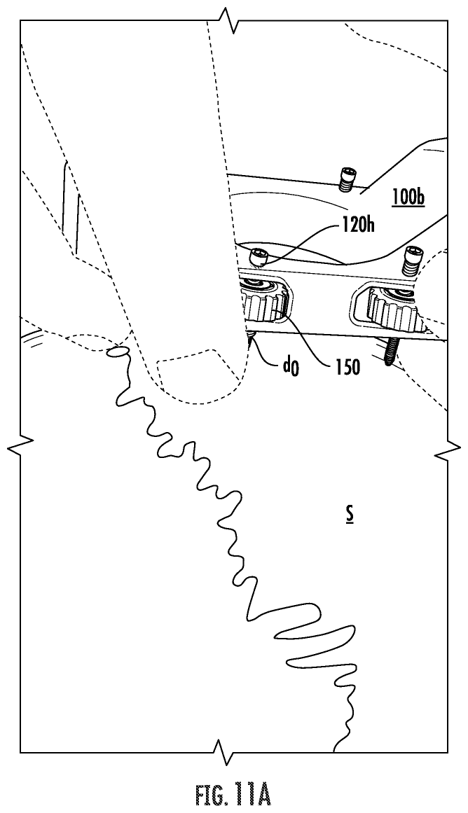

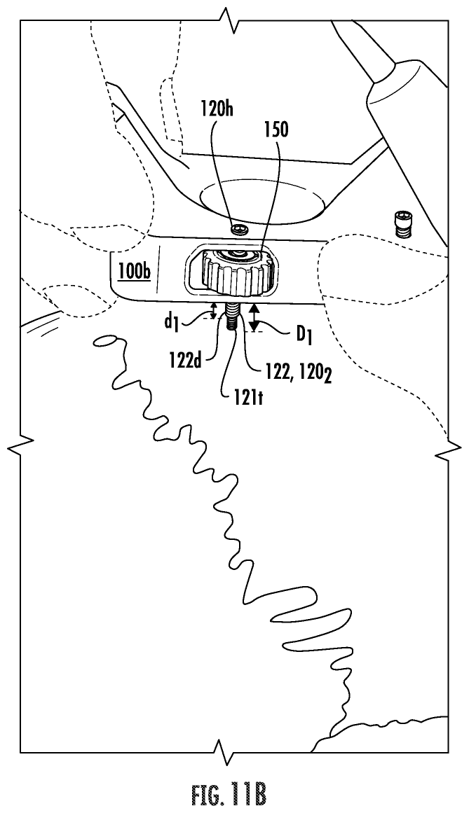

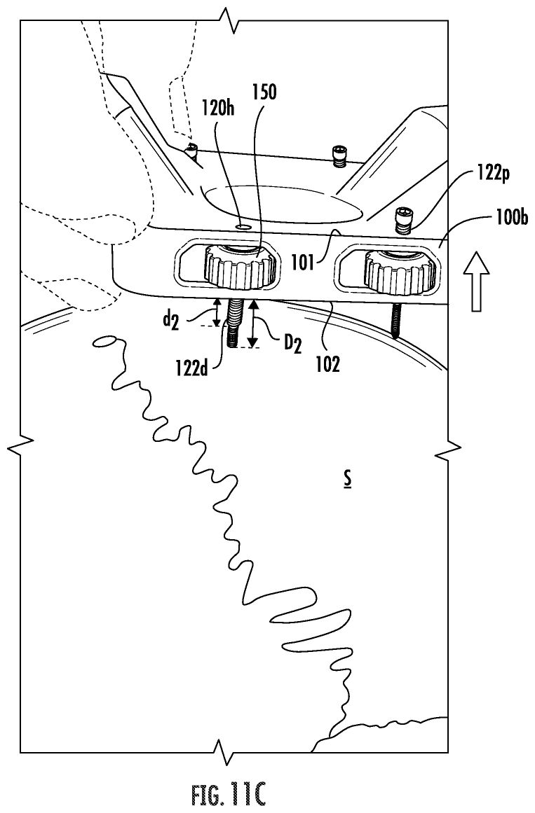

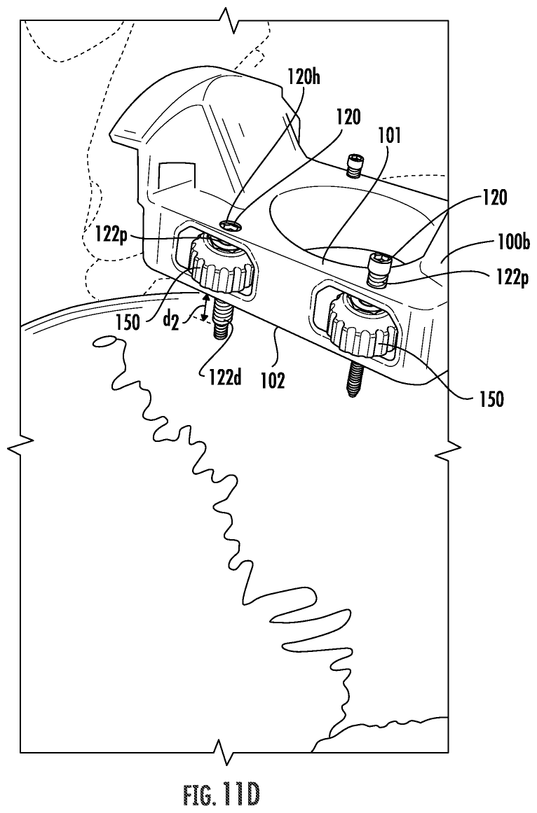

Turning now to FIGS. 10A-10C and 11A-11D, example actions are shown that can be used to attach the base assembly 100 to a patient for use in a surgical procedure, such as to mount the trajectory guide assembly 200 thereto for an image guided surgical procedure. The base assembly 100 can be secured to a patient before or after the trajectory guide assembly 200 is secured to the base assembly 100.

FIGS. 10A-10C illustrate a series of Step 1 actions using a driver 1303. In a start, pre-attached configuration, as shown in FIG. 10A, a proximal end 122p of the intermediate segment 122 with the external threads 122t can reside above a top surface 101 of the base assembly 100 and a small length of the distal end 122d of the intermediate segment 122 with the external threads 122t may reside adjacent the bottom 102 of the base assembly 100. A driver 1303 engages the head 120h of the threaded member 120 and rotates and drives the threaded member 120 so that the tip 120t enters the skull S.

As the driver 1303 drives the tip 120t threaded member 120 deeper, it drives the threaded segment 121t further into the skull S and causes the threaded segment 121t to self-tap into the skull S with the tip 120t of the threaded member 120 residing deeper into the skull S (compare FIG. 10A to FIG. 10C, for example).

Once the threaded segment 121t is secured to the scalp (FIG. 10C), the driver 1303 is no longer required. Instead, for Step 2, a user rotates the thumbwheel 150 (FIGS. 11A-11C) to lift the base assembly 100 away from the tip 120t of the threaded member 120 to a stabilized position (FIG. 11D). The steps can be repeated for the other threaded members 120 of the base assembly 100 providing four secure attachment points/regions, in the embodiment shown.

FIGS. 11B and 11C illustrate that as the thumbwheel 150 rotates, engaging the threads 122t of the intermediate segment 122, the base assembly 100 moves upward exposing a greater length of threads 122t at the distal portion 122d of the intermediate segment 122. This exposed length of the external threads 122t, labeled as d1 then d2, positions the bottom 102 of the base assembly away from the skull S a distance D1, then D2, respectively. Concurrently, the proximal end 122p of the intermediate member 122 may move down into the channel 151c (FIG. 4) under the head 120h so that the proximal end 122p of the threaded segment 122 is inside the nut 155 and no longer externally visible from the base assembly 100 and/or so that the head 120h is flush, or even recessed with the top surface 101 of the base assembly 100 (FIGS. 11C, 11D).

The movement of the base assembly 100 relative to the threaded member 120 causes the exposure of threads 122t at the distal end 122d of the threaded intermediate segment 122 to increase in exposed length from a start position of Step 2, do, shown in FIG. 10C/11A, to the stop position d2 of FIGS. 11C, 11D. The exposed length at a final stop position can be in a range of about 0.1 inches to 2 inches, more typically about 0.25-2 inches. The position of the bottom 102 of the base assembly 100 above the skull S at the stop position D2 can define the stand-off position ds (FIG. 7A, for example).

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A base assembly of a surgical trajectory guide frame, comprising:
a base body configured to hold a trajectory guide frame, the base body defining an open center patient access aperture;
at least one user-input actuator held by the base body; and
at least one threaded member, wherein one of the at least one threaded member extends through a respective one of the at least one user-input actuator.

2. The base assembly of claim 1, wherein the at least one threaded member comprises a first threaded section of a first diameter and a second threaded section of a second diameter that is greater than the first diameter.

3. The base assembly of claim 2, wherein the second threaded section threadably engages a respective user-input actuator.

4. The base assembly of claim 1, wherein the base body comprises spaced apart upper and lower primary surfaces, a first aperture in the upper primary surface and a second aperture in the lower primary surface, wherein the at least one user-input actuator is held between the upper and lower primary surfaces with an outer perimeter segment thereof extending outward a distance from the base body, and wherein the at least one threaded member resides through the first aperture, then through a center open channel of the at least one user-input actuator, then through the second aperture.

5. The base assembly of claim 1, wherein the at least one user-input actuator is a plurality of spaced apart user-input actuators, and wherein the at least one threaded member is a plurality of threaded members, one extending through each of the user-input actuators.

6. The base assembly of claim 1, wherein the at least one user-input actuator is a thumbwheel.

7. The assembly of claim 1, wherein the at least one threaded member comprises a head, an intermediate segment with external threads, and a shaft segment under the intermediate segment that merges into a lower end portion comprising external threads and a sharp tip configured to self-tap into bone.

8. The base assembly of claim 1, further comprising a plurality of nuts, one nut coupled to each of the at least one user-input actuator, the nut comprising internal threads that engage external threads of a respective threaded member whereby the base body is movable to a stand-off position while the threaded member remains in a fixed position and coupled to the base body.

9. The base assembly of claim 1, wherein the base body comprises an open center patient access port, and wherein the at least one user-input actuator is a plurality of user-input actuator residing at spaced apart locations of the base body.

10. The base assembly of claim 1, wherein the at least one user-input actuator is a plurality of user-input actuators, wherein each of the plurality of user-input actuators comprise a center through channel with a first segment residing under an upper primary surface of the base body that has a larger diameter than a second segment residing beneath the first segment, and wherein the base assembly further comprises a plurality of nuts with internal threads, one nut held inside the first segment of each of the plurality of user-input actuators.

11. The base assembly of claim 7, wherein the shaft segment is devoid of threads and extends a distance beneath the intermediate segment.

12. The base assembly of claim 1, wherein the base body is a monolithic molded body that is non-ferromagnetic, optionally wherein the at least one threaded member is a monolithic body of non-ferromagnetic metal.

13. The base assembly of claim 12, wherein the base body comprises a pair of yoke arms and an open center patient access port, and wherein the pair of yoke arms are spaced apart on opposing sides of the patient access port.

14. A trajectory guide system, the system comprising:
a base assembly comprising a base body coupled to a plurality of threaded members and a plurality of thumbwheels, one threaded member of the plurality of threaded members extending through one thumbwheel of the plurality of thumbwheels, wherein the thumbwheels threadably engage the threaded members; and
a trajectory guide coupled to the base assembly, wherein the trajectory guide is operable to move relative to the base assembly to position a trajectory axis to a desired intrabody trajectory to guide placement of a surgical device in vivo.

15. The system of claim 14, wherein each of the plurality of threaded members comprise first and second longitudinally spaced apart threaded segments of different diameters.

16. The system of claim 15, wherein each of the threaded members comprise a head that merges into an intermediate segment with external threads that merges into a shaft segment that merges into a lower end portion comprising external threads and a sharp tip.

17. The system of claim 14, further comprising a plurality of nuts, one coupled to each of the threaded members, the nut comprising internal threads that engage external threads of a respective threaded member whereby the base body is movable to a stand-off position while the threaded member remains in a fixed position and coupled to the base body.

18. The system of claim 14, wherein the plurality of threaded members is four, wherein each threaded member has a first segment of a greater diameter than a second segment and each of the first and second segments comprises external threads, and wherein each threaded member has a sharp tip configured to pierce through a scalp.

19. The system of claim 14, wherein the thumbwheels cooperate with the threaded members to provide a stand-off distance of the base body that is selectively adjustable relative to a distal tip of the threaded members.

20. A method for mounting an image-guided component on or adjacent a subject, the method comprising:
providing a base assembly with a plurality of spaced apart threaded members and a plurality of user-input actuators;
driving a head of each of the threaded members to extend the threaded members to a first position outside a bottom primary surface of the base assembly; and
rotating the user-input actuators about the threaded members to lift or lower the base assembly relative to the subject to a stand-off position while the extended threaded members remain in the first position.

21. The method of claim 20, wherein the base assembly comprises a center access port and pockets in the base body of the base assembly holding the user input actuators, and wherein the user input actuators are thumbwheels.

22. The method of claim 20, further comprising attaching a trajectory guide to the base assembly before, during or after the rotating.

23. The method of claim 20, wherein the threaded members comprise external threads having a first diameter proximate a sharp tip on distal end portion opposing the head, wherein the threaded members comprise an intermediate segment with external threads of a second diameter that is greater than the first diameter, and wherein the rotating the user-input actuators is carried out by rotating the user-input actuators about the external threads of the intermediate segment to lift the base assembly.

24. The method of claim 23, wherein the driving is carried out to self-tap a distal end portion of the threaded members into the subject to define the first position, wherein the rotating is carried out while the sharp tip is anchored in the subject.
